(19) United States
(12) Patent Application Publication
(10) Pub. No.: US 2025/0195179 A1
(43) Pub. Date: Jun. 19, 2025

(54) lmplant

(71) Applicant: Cudeti SAGL, Lugano (CH)

(72) Inventors: Marco Ravanello, Gentilino (CH); Bernard Touati, Neully (FR); Gianpaolo Savio, Padova (IT)

(73) Assignee: Cudeti SAGL, Lugano (CH)

(21) Appl. No.: 18/834,080

(22) PCT Filed: Jan. 27, 2023
(86) PCT No.: PCT/EP2023/052080

§ 371 Date: Jul. 29, 2024

(30) Foreign Application Priority Data
Jan. 28, 2022 (EP) 22305096.4
Mar. 24, 2022 (GB) 2204123.0

(51) Int. Cl. A61C 8/00; B33Y 80/00

(52) U.S. Cl. CPC A61C 8/0037; A61C 8/0022; A61C 8/009; A61C 2008/0046; B33Y 80/00

(57) ABSTRACT

There is described a medical implant comprising: a bone engaging portion comprising: an outer portion comprising or consisting of a conformal lattice structure; an inner portion comprising a cavity or a lattice structure; a helical thread extending around and recessed into or protruding from an outer surface of said bone engaging portion, said helical thread being configured to facilitate screwing of said bone engaging portion into bone.

### FIELD

The present disclosure relates to an implant—such as a medical or surgical implant—that can be inserted into, retained by and integrated with bone. In some embodiments, the present disclosure relates to a dental implant.

### BACKGROUND

Implants are used in human or animal bodies to treat orthopaedic trauma and disease around the body, such as in joint replacement, dental implants and fracture repair. These implants are used in high volume. Implant fixation is critical to the success of these procedures and loosening is often a primary reason for failure. Modern implants typically rely on press-fit or screw fixation. Screw fixation offers high levels of implant stability with high pull-out and push-in loads. This may be termed primary stability and is achieved by rotation and compression of the thread towards the bone.

Dental implants can be surgically implanted into a human or animal subject's jawbone to support an artificial tooth and restore chewing function. The artificial tooth is typically a prosthesis—such as a crown. Dental implants typically have a longitudinally extending distal portion with a standardised cross-sectional area and a standardised length. Often the implant is either threaded or press-fit into a void or bore which is drilled into the human or animal subject's mandible or maxilla jawbone at an edentulous site. The press-fit implant is usually inserted by applying a rotary push force to the implant in an insertion direction. For a threaded implant, self-tapping threads can be used for initial stability of the implant immediately after surgery. Before biological or secondary integration has time to take place, the threads can help to resist tension, twisting, pulling, pushing or bending loads applied to the implant.

One example of an implant is described in WO2018/011604. This implant utilises, inter alia, a conformal lattice structure design on the outside part of the bone engaging portion of the implant to improve stability by encouraging osseointegration with bone. Osseointegration is known as the structural and functional connection between living bone and the surface of a load-bearing implant. Since lattice structures comprise voids, they can encourage cell growth in the spaces of the lattice that can lead to osseointegration. Lattice structures also increase surface area thereby allowing for an increased potential for osseointegration. Thus, one way in which the fixation of implants can be improved is by osseointegration whereby bone integrates with the implant, thereby helping to hold it in place. However, osseointegration is not straightforward to achieve, takes time and is not always effective.

There is a continuing need in the art for an improved implant that provides a better, faster and higher chance of healing. There is also a requirement for an implant that is held firmly, blocked or anchored 3-Dimensionally so that micro movements are avoided or at least reduced, thereby improving the stability and longevity of the implant. The present disclosure seeks to address these and other needs.

### SUMMARY

According to exemplary embodiments of the present disclosure, the degree of osseointegration and osseopenetration that can be achieved with an implant is increased because there is more opportunity for these biological processes to occur not only with the outside of the implant but also with the inside of the implant. This is possible because biological material is able to access, and is encouraged to access, the inside of the implant. Since excellent osseointegration and osseopenetration with bone can occur both outside and inside the implant, it is expected that stronger integration with bone can be achieved leading to a more stable implant. This is despite the presence of less titanium (or other material) being present in the implant itself. The presence of less titanium (or other material) in the implant itself can be highly advantageous as the implant represents less of a foreign body in a subject into which the implant is inserted. Thus, stronger integration with bone may be achievable whilst reducing the risk of complications through rejection. Embodiments seek to increase both the speed and effectiveness of osseointegration and osseopenetration of an implant.

A first aspect provides a medical implant comprising: a bone engaging portion comprising an outer portion comprising or consisting of a conformal lattice structure, an inner portion comprising a cavity or a lattice structure; and a helical thread extending around and protruding from an outer surface of said bone engaging portion.

The helical thread is configured to facilitate screwing of said bone engaging portion into bone. In some embodiments the helical thread is configured to encourage the capturing and/or grating of biological material (such as bone cells, blood, bone chips and the like) inside the implant. The helical thread also acts to compress the biological material during insertion of the implant, thereby encouraging the ingress of the biological material into the cells of the lattice structure.

The present disclosure provides a medical implant with a bone engaging portion whose outer portion comprises or consists of a conformal lattice structure, which is a topologically ordered, three-dimensional open-celled structure composed of one or more repeating unit cells. These cells are defined by the dimensions and connectivity of their constituent strut elements, which are connected at specific nodes. The conformal lattice structure allows biological matter—such as bone cells, blood, marrow, bone chips and the like—to travel into the implant as the implant is screwed into bone. The passage of biological matter into the conformal lattice structure favours an osseopenetrating effect and improves secondary stability, via osseointegration and osseopenetration and reducing or inhibiting micro-movements of the implant. In particular, the presence of biological matter within the conformal lattice structure and implant improves the stability of the implant by reducing or inhibiting both lateral, longitudinal and rotational movement. The conformal lattice structure allows biological matter to travel and be retained within the conformal lattice structure. The presence of an internal portion that is either hollow or formed of a lattice structure that may be a conformal, non-conformal or random lattice structure allows the biological matter to travel beyond the conformal lattice structure of the outer portion and into the inner potion, thereby further promoting osseointegration and osseopenetration. Over time, the biological material colonises the lattice structure(s) of the implant thereby leading to osseointegration and osseopenetration of the implant within the subject. This helps to hold the implant firmly in position and helps it to become integrated within the bone material into which it extends. Furthermore, the osseointegration and osseopenetration provides structural support and robustness to the implant.

A conformal lattice structure comprises nodes and a set of struts that connect the nodes to form a plurality of cells, said plurality of cells forming a mesh. The cells may have different shapes and in some embodiments the cells are hexahedral. The conformal lattice structure is configured such that the cell sizes and shapes conform to the desired shape of the implant, allowing an implant to be manufactured that is adapted to a patient and yet has a mechanically very robust structure. Weaknesses in the structure that might arise due to the cells of a lattice structure being truncated to adapt to a certain size or shape are avoided or at least reduced in a conformal lattice structure by adapting the shape and size of the cells themselves. Advantageously, this allows an implant with a cellular outer portion to be provided that is both mechanically robust and allows osseointegration and osseopenetration.

A helical thread helps to hold the implant in position, particularly when it is first inserted. The helical thread protrudes from the implant. The helical thread also aids insertion of the implant, rotational movement and compression of biological material which encourages (for example, by scooping, pushing, grating and/or scraping) the migration of biological material into and through the cells of the conformal lattice structure on insertion and through to the inside of the implant.

The helical thread may be attached (for example, glued) to the outer surface of the implant or it may be formed from an extension of the conformal lattice structure, thereby being an integral part of the implant. Where it is formed as an extension of the conformal lattice structure, the walls of the cells of the conformal lattice structure may extend to form a solid thread such that the walls transition from wall to solid thread in a continuous manner. Not only does this provide a mechanically robust thread, but also the structure is such that cell openings of the conformal lattice structure are formed (immediately) before the thread in the direction of insertion so that the thread encourages (for example, by scooping, pushing, grating and/or scraping) the biological material into the cell openings.

In some embodiments, said bone engaging portion comprises an outer portion and an inner portion. The inner portion may be a cavity that is completely hollow.

An inner cavity within the implant may make insertion of the implant easier and improve osseointegration and osseopenetration with the presence of biological matter within the conformal lattice structure and inner cavity improving the stability of the implant and increasing the Bone Implant Contact (BIC) area. In this regard, there is a requirement for an implant to be strong and robust to cope with the extreme forces that are encountered when an implant is screwed into bone. There is therefore a technical prejudice against a hollow implant and particularly against combining a hollow implant with an outer portion that has a conformal lattice structure. The use of a conformal lattice structure allows mechanical strength to be designed into the implant by adapting the shapes of the cells of the conformal lattice structure to both accommodate the required shape of the implant and maintain mechanical strength and stability, thereby providing an improved implant that is strong, robust and aids osseointegration. The osseointegration and osseopenetration can reduce any micro movements of the implant and leads to a stable implant.

Advantageously, the extreme forces (which are mainly frictional forces) generated when an implant is screwed into bone are harnessed by helping to move biological matter into the implant which then promotes osseointegration and osseopenetration.

Suitably, said helical thread extends from said outer surface and has a solid form. The solid form is not a lattice structure or a conformal lattice structure. The outer portion and/or helical thread can be formed from titanium or alloys thereof or ceramic or tantalum or a combination of one or more thereof. The choice of material used to make the helical thread and the outer surface can be the same or different. In one embodiment, the choice of material used to make the helical thread and the outer surface is titanium or an alloy thereof.

Suitably, said conformal lattice structure comprises a plurality of cells and said helical thread is formed from an extension or continuation of some of said cells of said conformal lattice structure as said helical thread extends away from said outer surface of said outer portion to form a solid helical thread.

The helical thread may therefore be formed as an extension or continuation of the conformal lattice structure, with the cells extending out from the outer surface to form a solid helical thread. This provides a robust helical thread that merges seamlessly with the conformal lattice structure. The implant may be manufactured by additive manufacturing techniques.

According to this embodiment, the helical thread is solid and is not made from a lattice or conformal lattice structure.

The provision of a helical thread onto a conformal lattice structure is technically challenging. The extension of the surfaces forming the conformal lattice structure to from the solid screw thread provides a simple yet robust solution to this technical challenge.

Suitably, a thickness of the helical thread is constant.

Alternatively, a thickness of the helical thread decreases from one end of the bone engaging portion to the other end of the bone engaging portion.

A helical thread with a reduced width close to an insertion end of the implant may make insertion of the implant into bone easier.

In some embodiments, the width of the thread and/or the conformal lattice structure may be customised to suit the bone mineral density and/or bone quality of a subject.

In some embodiments, the width of the thread and/or the conformal lattice structure may be customised to suit the bone mineral density and/or bone quality of the subject and may vary along the length of the implant in accordance with variations in the subject's bone density.

Thus, a size of one or more cells within said conformal lattice structure is adapted to the bone mineral density and/or bone quality of a subject.

Suitably, the outer portion and/or the helical thread comprises or consists of ceramic materials.

Suitably, the outer portion and/or the helical thread comprises or consists of titanium or alloys thereof or tantalum or a combination thereof. Suitably, the titanium alloy is titanium aluminide.

Ceramic materials and the like are strong materials that do not corrode and which are biologically compatible and can help to further encourage osseointegration.

The conformal lattice structure encourages osseointegration and osseopenetration by the ingress of biological material into the cells of the conformal lattice structure during insertion and when in place. This can be facilitated where the cells of the conformal lattice structure are configured in dependence upon the mineral bone density of the subject. In this regard, when the bone is softer, larger cells may be appropriate, whereas, when it is harder then smaller cells may be preferred.

In some embodiments, said cells of said conformal lattice structure in at least an outer surface of said bone engaging portion are orientated such that insertion of said medical implant by screwing said implant into said bone facilitates the ingress of biological matter into said conformal lattice structure and inside the implant.

The provision of a conformal lattice outer surface not only provides additional surface area for contact between the implant and the bone (bone implant contact area (BIC), which is one of the most decisive factors for correct and stable bone integeration and secondary stability), but also provides passages for the ingress of biological material into the implant, thereby aiding osseointegration and osseopenetration. Having a thread on the outer surface of the implant means that it is inserted with a screwing action. With suitable configuration of the cells of the conformal lattice structure in at least the outer surface, a self-priming action may be attained when screwing the implant into a patient such that the screwing action pushes biological material into the suitably orientated cells on the outer surface and then through into the implant itself, there thereby facilitating the ingress of biological material and accelerating the processes of ossseointegration and osseopenetration.

In some embodiments, at least some apertures formed by said cells in said outer surface of said bone engaging portion are orientated to face towards a direction of insertion of said implant, such that insertion of said implant drives biological matter into said at least some apertures. In certain embodiments, all apertures formed by said cells in said outer surface of said bone engaging portion are orientated to face towards a direction of insertion of said implant.

Facing the apertures in the outer surface of the implant towards the direction of insertion of the implant allows the driving force for pushing the implant into the bone to also act to push biological material through the apertures and into the conformal lattice structure. The direction of insertion is towards an insertion end of the implant and also towards the rotational direction of insertion which is determined by the helical thread.

In some embodiments, the cells arranged adjacent to and on a side of said helical thread towards an insertion end of said implant, are configured such that apertures formed by said cells face towards a direction of insertion, such that biological matter compressed by said helical thread on insertion of said insert is pushed into said apertures.

The presence of the helical thread compresses biological matter adjacent to the thread and on the insertion side of the thread on insertion of the implant. This provides an additional force on the biological material and with suitable orientation of apertures adjacent to the thread this additional force can be used to encourage the ingress of biological material into the implant. Furthermore, in embodiments where the thread is formed from an extension of walls of the cells, this provides not only a mechanically robust structure, but also provides apertures in the cells adjacent to the thread and aligned to receive the biological material.

In some embodiments, at least some apertures formed by said cells in said outer surface are configured such that a portion of a perimeter of said at least some apertures are more remote from an insertion end of said medical implant and extend radially outwards further than a portion of said at least one aperture towards said insertion end.

One way in which the cells may be orientated to aid the ingress of biological material into the implant is by orientating the cells so that an aperture formed in the outer surface by the cell is angled so that it is not parallel with an axis of the implant but is angled so that it faces towards the insertion end, such that biological material is pushed into the aperture as the implant is inserted, during use, by rotation.

In addition to or as an alternative, at least some apertures that are angled with respect to the longitudinal direction of insertion may be angled with respect to the direction of rotation of the helical thread, such that the portion of the perimeter of the aperture that follows on rotation extends radially beyond the portion that leads allowing the aperture to scoop biological material into the cell.

In some embodiments, said cells are configured such that struts forming walls of said cells surrounding said at least some apertures on said outer surface are configured to provide angled surfaces to provide a grating or scraping effect when said implant is screwed into a bone.

The accumulation of biological material within the implant may be further aided by providing angled edges to the cells such that a grating or scraping effect is provided. This grating or scraping effect can release biological material which then finds its way into the implant.

In some embodiments, said conformal lattice structure is configured such that a size of cell within said conformal lattice structure varies along a length of said bone engaging portion, from a maximum at one end to a minimum at another end.

Varying the cell size along the length of the implant may have advantages. In particular, it may be advantageous for the cells to be larger towards the insertion end, encouraging more biological material to be retained in larger cells, and smaller towards the other end, where the implant may be more robust and retain less biological material.

Suitably, the conformal lattice structure is configured such that a thickness of cell struts or walls within the conformal lattice structure varies along a length of the bone engaging portion, from a maximum at one end to a minimum at another end.

Similarly, there may be advantages to varying the thickness of cell struts or walls to suit the application and/or the subject. Furthermore, varying their size along the length of the implant may also have advantages. In particular, it may be advantageous for the cell struts or walls to be thinner towards the insertion end and thicker towards the other end, increasing the void fraction of the implant close to the insertion end where more biological matter may be retained, and more robust towards the other end. This also favours the intake of biological material during rotational placement inside the bone.

In other embodiments, said conformal lattice structure is configured such that a size of cell within said conformal lattice structure is substantially constant along a length of said bone engaging portion.

Suitably, a void fraction of said conformal lattice structure varies along a length of said bone engaging portion from a maximum at one end of said bone engaging portion to a minimum at the other end of said bone engaging portion.

In other embodiments, a void fraction of said conformal lattice structure is substantially constant along a length of said bone engaging portion from a maximum at one end of said bone engaging portion to a minimum at the other end of said bone engaging portion.

In some embodiments, a stiffness of said medical implant is higher at one end of said bone engaging portion than at the other end.

In some embodiments, said inner cavity comprises between 40% and 60% of the volume of said bone engaging portion.

The inner portion can be hollow (meaning that it has only empty space inside) or it can contain a lattice structure that is regular or random or non-conformal or conformal. When the inner cavity contains the lattice structure it may be completely or partially filled with the lattice structure. In embodiments, when the inner cavity contains the lattice structure it is preferred that the inner portion is completely filled with the lattice structure. In embodiments where the inner portion of the implant is formed of a lattice structure, the lattice structure may be a random lattice structure. In other embodiments, the random lattice structure is configured to match a bone density of a patient.

The size of the inner cavity can be selected according to the properties required for the implant. In this regard, a thicker outer portion may form a more robust implant but may make it more difficult to insert and may impede improved osseointegration. It is found to be particularly effective to have an inner cavity that comprises between 40 and 60% of the volume of the bone engaging portion.

Suitably, the inner cavity has a circular cross section.

Alternatively, the inner cavity has a polygonal cross section.

Alternatively, the inner cavity has an elliptical cross section.

The inner cavity may have a number of different shapes. A circular shape is a preferred shape. However, in some cases a polygonal or an elliptical cross section may be advantageous, particularly where it may be preferable to provide a different shaped implant due to the bone into which it is to be inserted. In some embodiments the inner cavity comprises a thread like shape that is configured to favour the ingress of biological material.

Suitably, a cross sectional area of the inner cavity decreases along a length of the inner cavity.

In some cases, it may be advantageous for the cross-sectional area of the inner cavity to become smaller along a length of the inner cavity. It may be advantageous for the inner cavity to be wider towards the end of insertion and to narrow away from the end of insertion allowing more biological matter to be collected towards the insertion end which will pass through a greater amount of bone.

Suitably, a perimeter of the outer surface of the bone engaging portion is substantially circular.

Suitably, the outer surface of the outer portion is substantially cylindrical.

A substantially circular perimeter and/or a substantially cylindrical medical implant may be advantageous as this is a strong shape and one that is easy to insert.

Suitably, a cross sectional area of the outer surface is substantially constant along a length of the implant.

Alternatively, the outer perimeter of the bone engaging portion reduces in length from a maximum at one end to a minimum at the other.

Although a cylindrical form to the medical implant may be robust and relatively easy to manufacture, in some cases it may be advantageous to have a more conical or frusto-conical form to the implant and in particular one which has a smaller cross section at an insertion end. This may facilitate insertion.

Suitably, the inner cavity extends to one end of the bone engaging portion, such that one end of the bone engaging portion is open.

It may be advantageous for the inner cavity to extend to one end of the bone engaging portion such that, on insertion, biological matter can move directly into the inner cavity easing insertion and helping hold the implant in position.

Suitably, the inner cavity extends to both ends of the bone engaging portion, such that both ends of the bone engaging portion are open.

In some cases, the implant may be used to attach between two bone structures and in such a case and indeed, in other examples, it may be advantageous to have the inner cavity extend to both ends of the bone engaging portion allowing the biological matter to pass completely through the implant.

Suitably, the at least one channel and the inner cavity are configured such that an end of the at least one channel opens at least partially into a cell of the conformal lattice structure.

Suitably, a profile of the exterior surface comprises curved edges.

Curved edges can favour hydrophilicity and attract the attachment of biological material.

Suitably, the medical implant is a dental implant comprising: the bone engaging portion; a transmucosal portion configured to engage marginal soft tissue, wherein the inner cavity extends longitudinally towards the transmucosal portion. Suitably, the inner cavity does not extend into the transmucosal portion.

Medical implants of the embodiments described herein are particularly suitable as dental implants where it is important that they are held firmly and stably in position and where the amount of bone matter into which they can be inserted is limited. In this regard, the placement of dental implants requires a proper fixation in the jawbone to provide initial stability. Furthermore, the lattice structured implant is less invasive as it has less material than a solid implant and as it is osseopenetrated it does not represent a foreign body inside the bone but rather a scaffold comprising, in some embodiments, a metallic titanium alloy (TiAl) structure impregnated with bone. Such mesh structures show a much higher secondary stability or bone integration and also increase bone resistance and strength as they provide bone with a reticular TiAl support increasing the rigidity of the bone. A conventional screw implant is considered a foreign body which generates primary stability in the bone solely via compression and secondary stability by solidification of the bone surrounding the implant also known as osseointegration. In orthopedic applications for example, such solid screw implants are used to join two or more items such as bones and once the joint is safe the screw implant is removed as its presence weakens the bone. With the implant described hereinsuch weakening is avoided or at least reduced as 50-60% less material can be used leading to, for example, 40% to 50% more bone supported by the structure of the lattice.

Suitably, the transmucosal portion comprises a recess in a proximal end of the transmucosal portion, the recess being isolated from the conformal lattice structure of the inner cavity by a separating wall.

Where the implant is a dental implant it may be advantageous to separate the transmucosal portion from the inner cavity as this may help isolate the inner cavity of the implant from the exterior and impede infection.

The transmucosal portion may have a recess in a proximal end which may receive an insertion tool for helping insert the dental implant into the bone. It may also later receive the crown.

Suitably, at least a portion of said lattice structure comprises a plurality of interlinked cells forming a mesh structure, said cells comprising a gyroid form. The lattice structure may be the conformal lattice structure of the outer portion and/or the lattice structure or the inner portion.

Suitably, at least a portion of said lattice structure comprises a plurality of interlinked cells forming a mesh structure, said cells comprising an octahedral form. The lattice structure may be the conformal lattice structure of the outer portion and/or the lattice structure or the inner portion.

Suitably, at least a portion of said helical thread comprises a plurality of recesses, said recesses being formed at different axial and circumferential positions in said thread. Suitably, said plurality of recesses are arranged to form at least one spiral groove running through said thread.

Suitably said spiral groove runs through said thread in a different rotational direction to said thread.

A second aspect provides a combination of a non-transitory data file and the implant of the first aspect for replacing a tooth or filling an oral cavity void of a subject, wherein the implant is manufactured by a computer-aided manufacturing process in accordance with information contained in the data file, wherein the data file comprises information relating to physical and/or biological characteristics of the tooth to be replaced and/or the void to be filled and/or the surrounding bone of the tooth or void.

Providing an implant using a computer-aided manufacturing process allows the implant to be customised to a patient. As the implant has a conformal lattice outer structure and a void or lattice interior, the customisation can be made both with regard to the size and shape of the implant and also to the size and shape of the cells and in some embodiments the size and shape of the cavity. The determined desired size and shape of the cells and/or cavity may depend on the bone mineral density or the bone quality of the subject into which the implant is to be inserted. This customisation can facilitate or improve osseointegration with the surrounding mandibular bone. Such customisation may be made using digital imaging—such as 3-dimensional digital imaging—to generate a data file, the data file being used during the computer-aided manufacturing process which may be an additive manufacturing technique to configure the implant.

In some embodiments, said data file comprises information relating to: (1) a geometrical area and alignment of the tooth to be replaced and neighbouring teeth or geometrical area and alignment of the void; and/or (2) bone mineral density and/or bone quality of a mandibular bone of the subject; and/or (3) height of a cortical bone of the subject.

A third aspect provides a method of manufacturing a medical implant according to a first aspect, comprising: determining the mineral bone density of a subject at a site of insertion of said medical implant; manufacturing by additive manufacturing techniques a medical implant comprising a bone engaging portion comprising or consisting of a conformal lattice structure and having a thread extending around an outer surface of said bone engaging portion, at least one of a void fraction and a cell size of said conformal lattice structure being selected in dependence upon said determined mineral bone density of said subject.

In some embodiments, said medical implant comprises a dental implant comprising said bone engaging portion and a transmucosal portion and in some embodiments an abutment portion, said method of manufacturing said dental implant as a single one piece dental implant.

Additive manufacturing techniques allow a dental implant of a desired size and complex shape to be manufactured as a single piece which has the advantages of robustness avoiding junctions between different parts, which may experience micro movements and also the ingress of foreign bodies which may result in infection. It also allows the implant to be customised to the patient both with regard to the size and shape of the natural tooth or void in situ into which it is to be implanted and also to the density of the bone mineral density or the bone quality of the subject into which the implant is to be inserted. This can facilitate or improve osseointegration with the surrounding mandibular bone.

Further particular and preferred aspects are set out in the accompanying independent and dependent claims. Features of the dependent claims may be combined with features of the independent claims as appropriate, and in combinations other than those explicitly set out in the claims.

Where an apparatus feature is described as being operable to provide a function, it will be appreciated that this includes an apparatus feature which provides that function or which is adapted or configured to provide that function.

### BRIEF DESCRIPTION OF THE DRAWINGS

Embodiments of the present disclosure will now be described further, with reference to the accompanying drawings, in which:

FIG. 1 shows an external view of a variation of a dental implant according to an embodiment (left-hand figure) and an internal section (left-hand figure) thereof;

FIG. 2 shows an external view of a variation of a dental implant according to a further embodiment (left-hand figure) and an internal section (left-hand figure) thereof;

FIG. 3 shows a photograph of a dental implant according to an embodiment;

FIG. 4 shows a cross section through a dental implant viewed from a distal end;

FIG. 5 shows microscopic analysis of a cross section of an implant of FIG. 1 osseointegrated and osseopenetrated into bone;

FIG. 6 shows microscopic analysis of a cross section of an implant of FIG. 2 osseointegrated and osseopenetrated into bone;

FIG. 7 illustrates a (a) regular, (b) conformal and (c) warped lattice structure and an example of a conformal lattice structure;

FIG. 8 illustrates different examples of a conformal lattice structure wherein the cells have a gyroid form;

FIG. 9 shows a recessed groove in the thread that provides a grater type function on insertion of said implant; and

FIG. 10 shows the arrangement of apertures in an external surface of an embodiment of a dental implant.

### DESCRIPTION OF THE EMBODIMENTS

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilised, and other changes may be made, without departing from the spirit or scope of the subject matter presented herein.

As used herein, the singular forms “a”, “an”, and “the” include both singular and plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The term “consisting of” means that additional components are excluded and has the recited elements only and no more.

Before discussing the embodiments in any more detail, an overview will first be provided.

Embodiments relate to a medical implant, which can be implanted into various parts of the human or animal body—such as bone, tendon, ligament, cartilage and the like. Exemplary medical implants include spinal fixators, bone fracture fixation plates, tendon repair anchors, ligament reconstruction fixation screws, chondral repair implants, total joint replacement implants and dental implants. Such implants are formed of a conformal lattice structure. In some embodiments the implants have an internal cavity—such a hollow internal cavity, or void, or chimney—surrounded by an outer portion comprising, consisting or consisting essentially of the conformal lattice structure. The internal cavity can be at least 50%, at least 60%, at least 70%, at least 80%, at least 90%, at least 95% or substantially the same length as the bone engaging portion. In some embodiments the internal cavity is open at the insertion end. Some embodiments relate to a medical implant having a screw thread—such as a helical screw thread. In some embodiments, the helical screw thread protrudes from the implant and is located on the outer surface of the implant.

Some embodiments relate to a medical implant having a screw thread, an outer portion having a conformal lattice structure and an internal cavity, suitably a partially or completely empty internal cavity. Others relate to a medical implant with no internal cavity as it is filled partially or entirely with a lattice structure that can be a conformal or non-conformal lattice structure, suitably, a non-conformal lattice structure. In other words, the lattice structure can be contained in what would otherwise have been the internal cavity such that the internal cavity is no longer hollow.

The medical and technical effect of embodiments relate to improved osseointegration and improved osseopenetration as biological material (for example, blood, cells, bone and marrow and the like) can access the inner cavity and the cells of the conformal lattice structure increase the Bone Implant Contact (BIC) area which can lead to improved healing time and to a stronger secondary stability of the implant. The hollow form of the implant consists in principle of two different areas, namely an outer portion or outside skin made out of the material of the implant forming a conformal lattice structure—such as titanium, titanium alloy, tantalum, ceramics and the like, as described herein—and an internal cavity, internal void or internal chimney, optionally containing a lattice structure.

The internal area can be a circular round shape; a multi angular shape—such as a triangle, a square a pentagon—and/or an elliptical shape or oval. All of the above-mentioned shapes can be cylindrical or conical. The size of the internal void structure as a proportion of the outer portion or skin can vary according to customer specific needs and to the final application of the implant. Generally, the ratio is in the region of 60% outer conformal lattice structure and 40% inner cavity.

A medical and technical effect of embodiments is the provision of improved osseointegration and improved osseopenetration as biological material (for example, blood, cells, bone, marrow and the like) can access the inside of the implant increasing the BIC area. This can lead to improved healing time and to a stronger secondary stability of the implant. The open end is arranged such that biological matter is pushed into the internal cavity as the implant is inserted into the bone.

Where the implant is hollow the outer portion of the implant is formed of a conformal lattice structure, whereas if it is not hollow the bone engaging portion of the implant is formed of a conformal lattice structure on the outside and a lattice structure that is conformal or non-conformal, suitably, non-conformal.

The conformal lattice structure and/or lattice structure can have a void fraction of between 5% and 95%, suitably, between 50% and 95%, suitably, between 65 and 90%.

The conformal lattice structure and/or (non-conformal) lattice structure can have a cell size of between about 10 μm and about 1500 μm, suitably, between about 100 μm and about 600 μm.

The parameters can be adjusted such that they are close to the characteristics of the bone into which the implant is to be inserted. For example, trabecular bone has a porosity of between 50% and 90% and a pore size of between about 200 μm and about 300 μm; cortical bone has a porosity of between 5% and 10% and a pore size of about 100 μm. Thus, in embodiments, the lattice structure has a void fraction of between 50% and 90% and a cell size of between about 200 μm and about 300 μm or a void fraction of between 5% and 10% and a cell size of about 100 μm—such as between about 90 and about 110 μm.

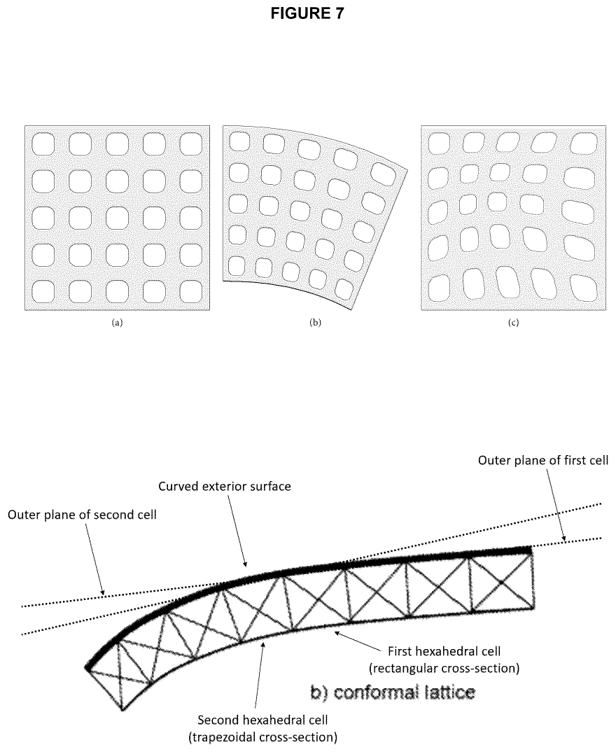

Lattice structures are generally described in Savio et al. (2018) Hindawi Applied Bionics and Biomechanics, 1-14 (the contents of which are incorporated herein by reference), which divides these structures into regular, pseudorandom, and random lattice structures. Regular lattice structures consist of a simple repetition of a unit cell such that the units cells are uniform in shape. Pseudorandom lattice structures are obtained by maintaining the topology and varying both size and geometry. These lattice structures can be further divided into warped and conformal lattice structures. Warped lattice structures are obtained by deforming the unit cell whilst keeping the original topology. In conformal lattice structures, the geometry and size of each cell is different in order to adapt (ie. conform) to its shape. Compared to regular lattice structures, conformal lattice structures never present interrupted or incomplete cells and have non-uniform cell shapes; this feature eliminates or at least reduces weakness at boundaries and provides stiffness and resistance. Conformal lattice structures comprise unit cells in which the geometry and size of the cells is different in order to adapt to a certain external topology, while the inherent strength of the lattice is substantially maintained. Compared to regular cellular materials, conformal ones never present interrupted or incomplete cells; this feature eliminates weakness at boundaries and provides stiffness and resistance. These different lattice structures are exemplified in FIG. 7.

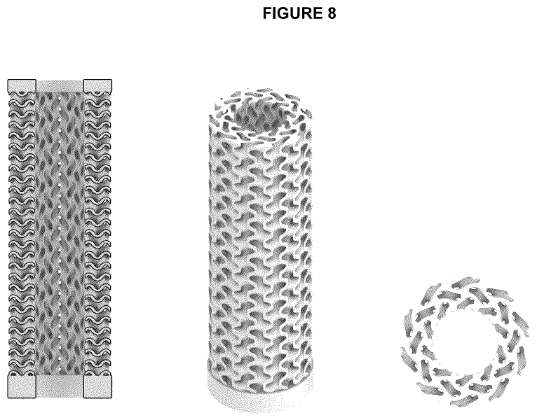

Different cell unit shapes are contemplated herein including square, hexahedron, triangular, tetrahedron, octahedron, rhombic dodecahedron, dodecahedron and iscosahedron. In certain embodiments, a hexahedral cell unit shape is preferred. In other embodiments gyroid shaped cells are preferred. FIG. 8 shows examples of gyroid shapes that may be used both in the conformal lattice structure and in the interior lattice structure, they have the advantage of curved or rounded surfaces which favour hydrophilicity and attract the attachment of cells and blood. Furthermore, such shapes form complex interconnecting passages that can be angled to favour the ingress of biological material as the implant is screwed into the bone.

According to example embodiments of the present disclosure, a lattice structure that is non-conformal can be a regular or a random lattice structure.

Conformal lattice structure design and fabrication is described in, for example, Solid freeform fabrication proceedings (2012), 138-161, Annual international solid freeform fabrication symposium, Texas, Austin (the contents of which are incorporated herein by reference). A conformal lattice structure is configured such that the cell sizes and shapes conform to the desired shape of the implant and the cells are non-uniform. This allows an implant to be manufactured that is adapted to a patient and yet has a mechanically robust structure.

According to the present disclosure, the structure of at least the outer portion is a conformal lattice structure. A conformal lattice structure comprises nodes and a set of struts that connect the nodes to form a plurality of cells, the plurality of cells forming a mesh. The cells in the conformal lattice structure are non-uniform and have different shapes. At least a portion of the mesh can be, for example, a hexahedral mesh formed of hexahedral cells, the mesh conforming to an exterior surface of the bone engaging portion. Computer-aided design technologies can be used for efficiently generating and representing conformal lattice structures. Software to achieve this is commercially available, for example, from Rhinoceros. Conformal lattice structures can be fabricated using additive manufacturing for the fabrication of customised, light-weight material. Software is generally available in the art for this process and can be integrated into a commercial CAD system, as required.

In this regard a conformal lattice structure as shown in FIG. 7 depicts a subset of the hexahedral cells that include cell-to-cell geometric variability so that the mesh conforms to a curved exterior surface of the longitudinally extending distal portion. As annotated adjacent cells that form the curved exterior surface are uncut (e.g., hexahedral cells) and have geometric variability to allow the cells to match the curvature of the exterior surface (e.g., “the first geometry of the first hexahedral cell is different than the second geometry of the second hexahedral cell such that the first outer plane is disposed at an angle relative to the second outer plane”).

In summary the conformal lattice structure comprises nodes and a set of struts that connect the nodes to form a plurality of cells, the plurality of cells forming a mesh, at least a portion of the mesh being a hexahedral mesh formed of hexahedral cells, and at least a subset of the hexahedral cells include cell-to-cell geometric variability so that the mesh conforms to a curved exterior surface of the longitudinally extending distal portion. The subset of the hexahedral cells includes a first hexahedral cell adjacent to a second hexahedral cell, the first hexahedral cell and the second hexahedral cell being disposed at the curved exterior surface of the longitudinally extending distal portion, the first hexahedral cell has a first geometry defined by a first set of nodes and a first set of distances between respective pairs of nodes of the first set of nodes, the first hexahedral cell having a first outer plane defined by outer nodes of the first hexahedral cell are disposed on the exterior surface, the second hexahedral cell has a second geometry defined by a second set of nodes with a second set of distances between respective pairs of nodes of the second set of nodes, the second hexahedral cell having a second outer plane defined by outer nodes of the second hexahedral cell that are disposed on the exterior surface, The first geometry of the first hexahedral cell is different than the second geometry of the second hexahedral cell such that the first outer plane is disposed at an angle relative to the second outer plane that matches a curvature of the curved exterior surface at the first and second hexahedral cells,

The term ‘additive manufacturing’ is used to describe the process of making a three-dimensional solid object by the laying down of successive layers of an extrudable and settable material from a moving dispenser. This technique allows an implant to be generated that is adapted for a subject and application. Scanning of the area in which the implant is to be placed can be used to determine the geometrical spaces and shape of the portions of the implant. It can also be used to determine mineral bone density. The implant may, for example, be shaped to be screwed into a void. In such a case, the implant can be manufactured slightly larger than the void, such that biological material at the edge is compressed along the outer portion or skin of the implant and forced into the implant. Additive manufacturing can be used to create lattice structures, which may have a void fraction that varies along one the length of the implant. The void fraction of the implant may also vary according to the application of the implant and to the subject. In effect the dimensions of the cells of the lattice, and the thickness of the material around the cells of the lattice may be adapted using additive manufacturing to provide an implant that has been customised or optimised for a subject or purpose. It may, for example, be advantageous for the cells of the lattice to be larger in some circumstances to increase contact surface area with the subject, and smaller in others to increase strength. Furthermore, the thread may have a width suited to the bone density of the subject.

The implant may therefore be made by additive manufacturing techniques-such as 3D printing.

The implant may be formed of ceramics, tantalum, titanium or alloys thereof. The thread may be formed of different materials or it may be formed of the same material. The thread may be formed as an extension of the walls or struts of the cells within the conformal lattice structure. The thread will not be made from a lattice structure but will be solid—such as solid ceramic, tantalum, titanium or alloys thereof.

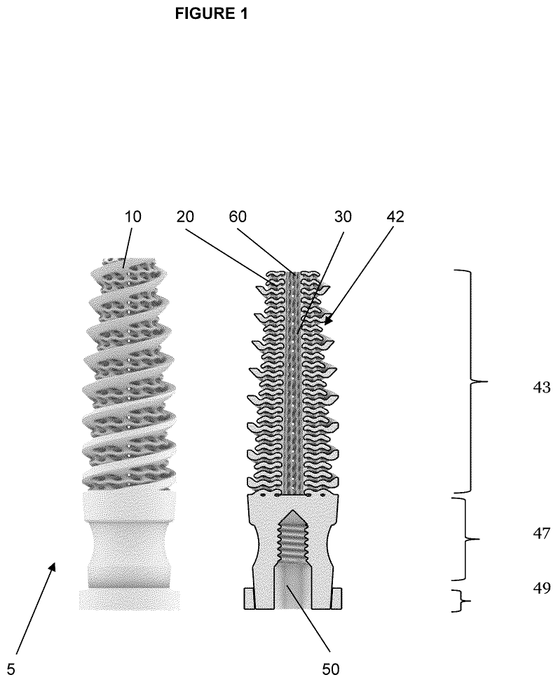

FIG. 1 shows a medical implant. In this embodiment, the medical implant is a dental implant 5 that comprises an outer portion or skin 20 formed as a conformal lattice structure and an inner portion 30 that in this embodiment is empty or hollow or void. In other embodiments, it may be filled with a lattice structure. The lattice structure of the inner portion may be a uniform lattice structure or a random lattice structure. The relative sizes of the outer lattice structure portion to the inner portion can vary according to specific needs and to the application of the implant. Generally, the ratio is of the order of 60% outer portion or skin to 40% inner cavity. This can provide an implant that is structurally robust and provides good osseointegration.

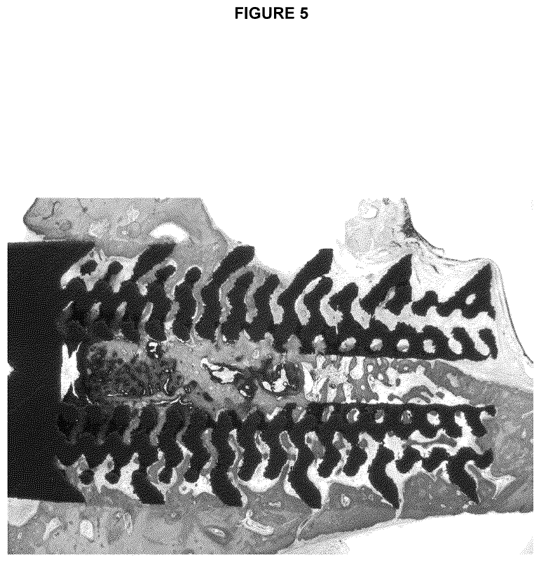

FIG. 5 shows microscopic analysis of a cross section of an implant of FIG. 1 osseointegrated and osseopenetrated into bone. In this embodiment, the outer portion 20 has a thread 10 running around the outer surface of the outer portion. The cavity 30 has an opening 60 (which can be a complete or partial opening but preferably a complete opening) at one end of the implant and provides a passage for biological material (for example, blood, cells, bone chips, marrow and the like) to access the inner cavity 30 of the implant. The conformal lattice structure of the outer portion 20 provides a volume through which the biological matter can migrate and ossify to hold the implant in place.

The size and shape of the cells of the conformal lattice structure and the thickness of the walls may vary and may be adapted for the subject and/or application. In this regard, it may be advantageous for the cells to be larger towards the insertion or distal end of the implant where they may retain more biological matter and smaller towards the other or proximal end. Similarly, the thickness of the thread may vary along the length of the implant.

The thickness of the struts or walls of the cells of the conformal lattice structure may also vary along the length of the implant. In this regard it may be advantageous to have a structure with a higher void fraction towards the insertion end as this end travels through more biological material and may retain more of such material. In some embodiments, due to the circular geometry of the implant the lattice may also have geometrical features that vary across the radius of the implant, whereby towards the outer part of the implant stud thickness and void area is larger, while towards the inner part they decrease.

In this embodiment, the insertion end has an aperture 60 providing access to the inner portion for biological material as the implant is inserted into the subject.

This embodiment comprises a helical thread 10 running around the exterior surface of the outside part of the bone engaging portion 43, which thread facilitates the screwing in of the implant into the bone and helps retain the implant in position. There is a transmucosal portion 47 that is configured to contact the soft tissue of the gum and an abutment portion 49 on which a crown may be mounted.

The dental implant of FIG. 1 comprises a recess 50 at the end of the transmucosal and abutment portions, remote from the insertion end which can be used to both receive an insertion tool for screwing the implant into the bone and in conjunction with the abutment portion receive a crown when the implant is in position.

The implant may be made of ceramics, or an alloy of tantalum or titanium and the like and it may be made by additive manufacturing techniques—such as 3D printing.

As can be seen, the struts or walls of the cells in the conformal lattice structure extend outwards to transition into forming the helical thread 10 running around the outer surface of the outer portion 20 that surrounds the cavity 30 of the dental implant 5. The helical thread is therefore formed from an extension of conformal lattice structures extending away from the outer surface of the outer portion 20 to form the thread. The walls of cells extend to form a solid thread. By virtue of this design, the conformal lattice structure has apertures 42 in the outer surface and these may be angled to encourage the ingress of biological material when inserted into the patient. In this regard, the apertures may be angled such that they are not parallel with the longitudinal axis but rather face towards the direction of insertion and the direction of rotation during insertion. In some embodiments, the edges of the cells of the conformal lattice structures may have be angled to provide a grater or scarping like effect on insertion. In FIG. 1, an aperture 42 is illustrated located between adjacent threads, such that compression of the biological material by the threads on insertion pushes the biological material into the aperture located at this point of compression and angled to receive the material as the implant is inserted. Apertures 42 of the conformal lattice structure are formed (immediately) in front of the thread in the direction of insertion so that the thread encourages biological material into the cell openings.

In summary, the presence after insertion of the implant, of biological material both within the conformal lattice structure and the inner cavity helps to maintain the stability and in particular, the secondary stability of the implant.

In these embodiments, the helical thread is shown protruding from the outer surface. Accordingly, the thread is formed from struts or walls of the cells that extend to form the helical thread. In this way a mechanically robust thread is provided.

Furthermore, the arrangement facilitates the thread scooping and pushing biological material into openings of the cells in the surface of the implant.

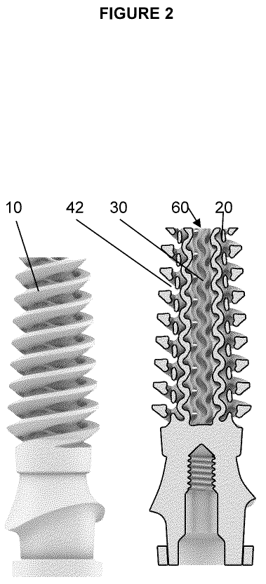

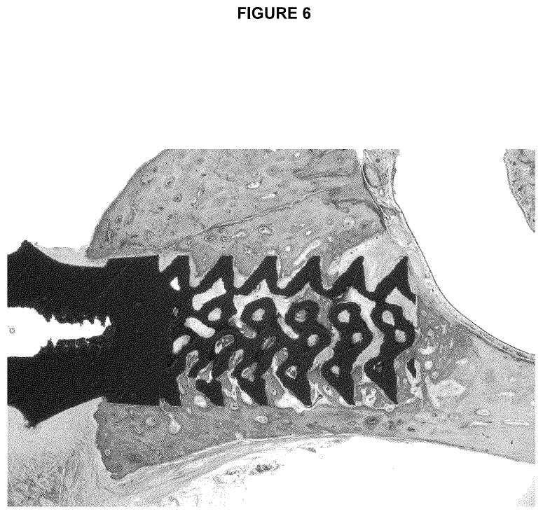

FIG. 2 shows a further embodiment of an implant with an outer portion 20, a thread 10 an opening 60 at the distal end, an interior hollow cavity 30 and apertures 42. This embodiment shows a differently configured conformal lattice structure, with different shaped and arranged cells. FIG. 6 shows microscopic analysis of a cross section of an implant of FIG. 2 osseointegrated and osseopenetrated into bone. The walls of the cells in the conformal lattice structure transition into forming the helical thread 10 running around the outer surface of the outer portion 20 that surrounds the cavity 30 of the dental implant 5. The walls of the cells form into a solid thread. This design also creates apertures 42 in the outer surface and these may be angled to encourage the ingress of biological material when inserted into the patient. In this regard, the apertures may be angled such that they are not parallel with the longitudinal axis but rather face towards the direction of insertion and the direction of rotation during insertion. Apertures 42 of the conformal lattice structure are formed (immediately) behind the thread in the direction of insertion so that the thread encourages biological material into the cell openings. In some embodiments, the edges of the cells of the conformal lattice structures may have sharp edges to provide a grater or scarping like effect on insertion. In FIG. 2, an aperture 42 is illustrated located between adjacent threads, such that compression of the biological material by the threads on insertion pushes the biological material into the aperture located at this point of compression and angled to receive the material as the implant is inserted.

As shown in FIGS. 1 and 2, apertures 42 are formed between each thread.

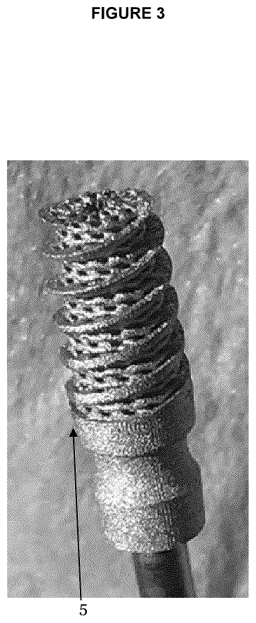

FIG. 3 shows an implant 5 according to an embodiment, with an open end and threads formed on a conformal lattice outer surface. The cells in the outer surface form apertures that are angled so that a following edge of the perimeter of the aperture when rotating the implant extends radially further than a leading edge. In some embodiments, an edge further from the insertion end may extend radially further than an edge remote from the insertion end. Furthermore, the edges forming the aperture may be sharp helping to cut through the biological material. In this way the edges of the aperture provide a grating or scraping effect on insertion of the implant and the orientation of the apertures help collect the biological fragments which are then pushed into the lattice structure by the movement of insertion.

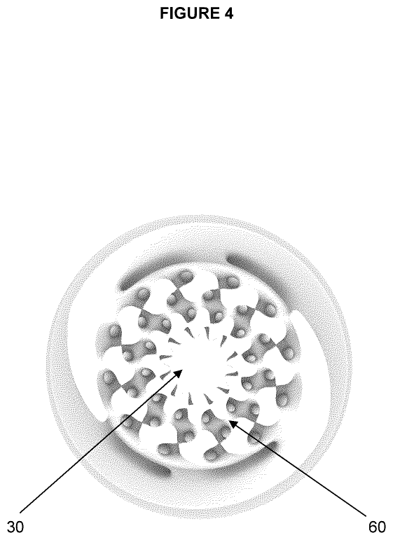

FIG. 4 shows an implant from the distal end, with an opening 60 to a hollow middle 30 and a conformal lattice outer portion.

The implant can be made of a suitable biocompatible material as described herein.

The different portions of the implant can be made from the same biocompatible material or different biocompatible materials. The different portions of the implant can be made from the same biocompatible material or different biocompatible materials that have a different surface finish. When the same biocompatible material is used, one portion of the implant can be made of a first biocompatible material and the other portion of the implant can be made from the same first biocompatible material. When the same biocompatible material is used, one portion of the implant can be made exclusively of a first biocompatible material and the other portion of the implant can be made exclusively from the same first biocompatible material. When different biocompatible materials are used, one portion of the implant can be made of a first biocompatible material and the other portion(s) of the implant can be made from a second, different biocompatible material(s). When different biocompatible materials are used, one portion of the implant can be made exclusively of a first biocompatible material and the other portion(s) of the implant can be made exclusively from a second, different biocompatible material(s). Alternatively, one portion of the implant can be made exclusively of a first biocompatible material and the other portion of the implant can be made exclusively from the same first biocompatible material.

The use of different combinations of biocompatible materials to manufacture the implant or one or more portions thereof is contemplated. The use of different combinations of biocompatible materials to manufacture two or more or three or more of four or more portions of the implant is contemplated.

The implant can be customised to correspond to the exact shape and dimensions of a human or animal subject's anatomy and/or biology. In other words, the implant can be a subject customised implant tailored to the subject into which the implant is to be inserted. For instance, the implant or one or more portions thereof may have dimensions, materials, and/or exterior surfaces that are configured to match the exact dimensions or requirements of a subject's anatomy. To accomplish this customisation, imaging technologies can be used to shape and size the implant or one or more portions thereof to correspond to subject specific anatomy and/or the implant or one or more portions thereof may have dimensions, materials, and/or exterior surfaces that are configured to match the bone mineral density and/or bone quality of a patient's anatomy. The implant can be a one-piece or two-piece or multiple piece implant, each part of the implant being customised to a patient's anatomy.

Whilst the implant described herein can be manufactured for use in various purposes as described herein, a preferred embodiment relates to a dental implant. The goal of a dental implant is to restore the human or animal subject to normal function, comfort, aesthetic, speech and health regardless of the current oral condition. A dental implant can allow a prosthesis—such as a dental crown—to be securely anchored to the bone. A precision fit of the dental implant is of the highest importance to reduce mechanical stress and enable good function and comfort for the human or animal subject following implantation.

In summary, there is provided implants adapted to a subject's anatomy.

Embodiments provide faster and safer osseointegration, the conformal lattice structure allows biological material (for example, cells, blood, marrow, bone chips and the like) to not only be compressed on the outside skin of the implant but allow them to penetrate inside the implant and thereby favour an osseopenetrating effect. This provides improved secondary stability, via this osseointegration and osseopenetration and can inhibit micro-movements of the implant.

Embodiments of implants have a conical or cylindrical geometry, a portion or all of which may comprise a conformal lattice structure to increase BIC on the surface area of the implant.

A helical projection—such as a thread—is provided for an improved stable coupling between the implant and bone.

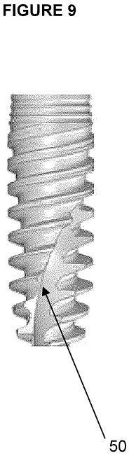

FIG. 9 shows a curved recessed groove 50 running through the helical thread along at least a portion of the length of the exterior surface of the implant that comprises the helical thread. The edges of the curved recessed groove 50 in the helical thread provide additional sharp edges that provide a grater type function when the implant is screwed into the bone and aids in the insertion process. Although one recessed groove 50 is shown in this embodiment, in other embodiments there may be a plurality of recessed channels. The recessed groove may run along the whole length of the threaded portion of the implant, or it may only run along a fraction or portion of the threaded portion. This Figure schematically shows the recessed groove and channels in the implant, the lattice structure of the outer surface is not shown.

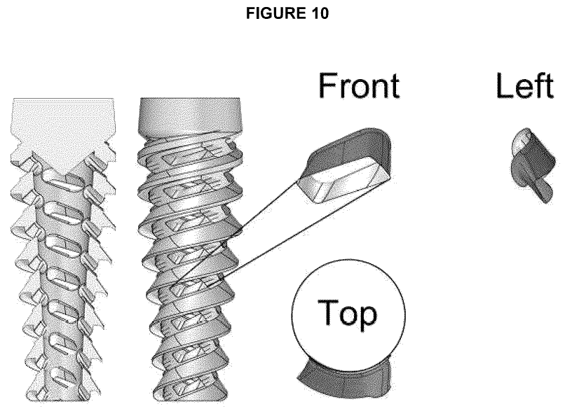

FIG. 10 shows the tilted non-circular apertures between the threads according to an embodiment, these provide a path orientated. The presence of the helical thread compresses biological matter adjacent to the thread and on the insertion side of the thread on insertion of the implant. This provides an additional force on the biological material and where apertures and channels are provided with suitable orientation of the apertures adjacent to the thread this additional force can be used to encourage the ingress of biological material into the implant.

One way in which the channels may be orientated to aid the ingress of biological material into the implant is by orientating the channels so that an aperture formed in the outer surface by the channel is angled so that it is not parallel with an axis of the implant but is angled so that it faces towards the insertion end, such that biological material is pushed into the aperture as the implant is inserted, during use, by rotation.

In addition to or as an alternative, at least some apertures that are angled with respect to the longitudinal direction of insertion may also be angled with respect to the direction of rotation of the helical thread, such that the portion of the perimeter of the aperture that follows on rotation extends radially beyond the portion that leads allowing the aperture to scoop biological material into the cell.

In some embodiments a method of manufacturing an implant is provided. This method uses additive manufacturing techniques—such as 3D printing—to produce the conformal lattice structure that forms at least the outer portion of the implant, which, in embodiments, can form the helical thread as an extension of the cell walls of the lattice structure. In some embodiments, the subject to receive the implant is scanned to determine the bone density at the intended site of the implant and the implant is then configured so that at least one of the conformal lattice cell size and thread thickness are adapted for that bone density. In some embodiments, these may change along the length of the implant where the bone density is detected as changing. Images of anatomical structures may be obtained using CB or CBCT based scanning technology that is known in the art. During imaging, the CBCT scanner rotates around the human or animal subject's head, obtaining numerous distinct images. The scanning software collects the data and reconstructs it, producing a digital volume composed of three-dimensional voxels of anatomical data that can be manipulated and visualized with specialised software. The scanning software can be used to determine bone mineral density and/or bone quality and/or the shape required for the implant. This can be provided in a data file and this data file may be used in the additive manufacturing technique to generate the implant. The data file can comprise or consist of information relating to: (1) a geometrical area and alignment of the tooth to be replaced and optionally neighbouring teeth; and/or (2) geometrical area and alignment of the void; and/or (3) bone mineral density of a bone and/or bone quality of the subject—such as mandibular bone; and/or (4) the height of a bone of the subject—such as cortical bone—of the subject; and/or (5) the thickness of the marginal soft tissue of the subject. The techniques can generate the implant as a one-piece implant so that the bone engaging and transmucosal pieces are formed as a single piece.

‘Bone quality’ generally encompasses factors including skeletal size, the architecture and 3-dimensional orientation of the trabeculae of bone, and matrix properties. Bone quality is a matter of mineral content and structure. The success rate obtained with the integration of implants typically depends on the volume and quality of the surrounding bone. In the case of dental implants, the surrounding bone of interest is mandibular bone. It is desirable to understand the bone quality of the surrounding bone so that the implant can be customised to the needs of an individual subject. Bone quality is well-known to be categorized into four groups: groups 1-4 or type I to IV: Type I is homogeneous cortical bone; Type II is thick cortical bone with marrow cavity; Type III is thin cortical bone with dense trabecular bone of good strength; and Type IV is very thin cortical bone with low density trabecular bone of poor strength. The implant of the present disclosure can be customised to the match the bone quality of the subject into which the implant is to be inserted.

Bone mineral density is generally defined as the amount of bone tissue in a certain volume of bone. Several methods are well known to measure bone density. For example, densitometric measurements of panoramic and periapical radiographs or more advanced methods—such as Dual Energy X-Ray Absorptiometry (DEXA), CT and CBCT can be used. In certain embodiments, it is preferred to use CT and/or CBCT. The implant of the present disclosure can be customised to the match the bone mineral density of the subject into which the implant is to be inserted.

The disclosure is further described in the Examples below, which are provided to describe the disclosure in further detail. These examples, which set forth a preferred mode presently contemplated for carrying out the disclosure, are intended to illustrate and not to limit the disclosure.

### EXAMPLES

### Example 1—Implantation of Implants into Pig Jaw

An implant according to FIG. 1 or an implant according to FIG. 2 is implanted into pig jaw using methods that are well known in the art. The placement of the implants is made according to one of two different methods:

In a first method, termed an extraction/implantation method, a tooth is extracted and simultaneously the implant is placed inside the edentulous area finding enough bone to primarily stabilize the implant. The bone is usually prepared meaning drilled with a pilot drill and subsequently a series of drills of progressive diameter are used in order to adapt to the size of the implant. It is common to have an interference fit between the drilled diameter and implant diameter in order to favour friction and achieve primary stability and anchorage.

In a second method, termed a healed site/implantation method, the tooth is extracted a few months before so that the dentulous area has been healing and has regained bone consistency and soft tissue coverage. In this instance the health care provider is preparing the area to be drilled by opening the soft tissue this can be done in 2 different ways by raising a squared flap or by punching a round circular into the soft tissue. As in the previously described procedure, a pilot drill is used to generate a hole to guide the progressive increase of the diameter of the drills. It is common to have an interference fit between the drilled diameter and the implant diameter in order to favour friction and achieve primary stability and anchorage.

Twelve weeks after implantation, the pig jaws are removed and sent to a histopathologist for analysis.

### Example 2—Preparation of Ground Sections for Light Microscopy

Using a bandsaw equipped with a diamond coated blade (Exakt, Norderstedt, Germany), the sections containing implants are cut out of the jaws under continuous cooling with tap water. The samples are then transferred in containers filled with 70% ethanol and evaluated by micro CT. The specimens are further dehydrated for approximately 4 days in each step in an ascending series of an ethanol-pure water series with the final step being in absolute ethanol (Sigma-Aldrich). The specimens are then infiltrated with a graded series of a Ethanol/Technovit 7200 VLC (Kulzer, Wehrheim, Germany) embedding resin over a period of at least 12 days at standard temperature while constant shaking. This is finished with the specimens being placed in 3 consecutive containers of 100% Technovit 7200 VLC for 24 h. Then, the specimens are placed into embedding moulds filled with fresh Technovit 7200 VLC and polymerized by 450 nm light for 10 hours, while cooling with running tap water to avoid temperature exceeding 40° C. Polymerized blocks are sliced in the bucco-lingual direction using an Exakt cutting unit (Exakt, Norderstedt, Germany). The slices are reduced by microgrinding and polishing using an Exakt grinding unit to an even thickness of 60-80 μm. Polishing is applied with 0.1 μm diamond paste (Struers DP-Paste M). Finally, the sections are stained with Sanderson's RBS (Dorn & Hart, Villa Park, US) and counter-stained with acid fuchsin. Sections are cover slipped for analysis using both a Leica M205A stereo light microscope and a Leica DM6B light microscope.

### Example 3—Preparation of Ground Sections for Back Scatter Scanning Electron Microscopy

Selected ground sections are prepared for Scanning Electron Microscopy. In short, after polishing, the section is sputter-coated with a 6 nm carbon layer and evaluated using a backscatter signal detector in a Zeiss 40 VP high resolution Scanning Electron Microscope.

### Example 4—Results Obtained Using the Implant of FIG. 1

The implant measures 4.3 mm in diameter and 8 mm in length. The inner portion has a hollow cavity.

The histopathology results based on the microscopic analysis show:
a. An osseointegrated implant with ingrowth of bone from both the lateral walls of the osteotomy as well as through the open inner channel. Ongoing bone formation was observed;
b. Ongoing bone formation by contact osteogenesis out from the lateral osteotomy wall;
c. Ongoing bone formation through the inner open channel; and
d. Ongoing bone formation by contact osteogenesis out from the lateral osteotomy wall.

There is penetration of bone inside the implant along with bone generation and acceptance.

FIG. 5 illustrates excellent osseointegration and excellent osseopenetration of the implant in bone.

### Example 5—Results Obtained Using the Implant of FIG. 2

The implant measures 4.3 mm in diameter and 8 mm in length. The inner portion has a hollow cavity.

The histopathology results based on the microscopic analysis show:
a. Osseointegrated implant. Inflammatory-free periimplant soft tissue. No coronal boneloss. Still ongoing bone ingrowth into the inner lumen;
b. Junctional epithelium adherent to the implant surface;
c. Inflammatory-free connective tissue opposed to the implant surface;
d. New woven bone growth into the lumen of the implant;

Ongoing bone formation despite the presence of connective tissue. Osteoblasts form the collagenous matrix of bone, the osteoid. The latter becomes mineralized to woven bone. Contact osteogenesis along the implant surface. There is penetration of bone inside the implant along with bone generation and acceptance.

FIG. 6 illustrates excellent osseointegration and excellent osseopenetration of the implant in bone.

Although illustrative embodiments of the disclosure have been disclosed in detail herein, with reference to the accompanying drawings, it is understood that the disclosure is not limited to the precise embodiment and that various changes and modifications can be effected therein by one skilled in the art without departing from the scope of the disclosure as defined by the appended claims and their equivalents. Any publication cited or described herein provides relevant information disclosed prior to the filing date of the present application. Statements herein are not to be construed as an admission that the inventors are not entitled to antedate such disclosures. All publications mentioned are herein incorporated by reference.

1. A medical implant comprising:
a bone engaging portion comprising:
an outer portion comprising or consisting of a conformal lattice structure;
an inner portion comprising or consisting of a cavity or a lattice structure;
a helical thread extending around and protruding from an outer surface of said bone engaging portion.

2. The medical implant according to claim 1, wherein said inner portion comprises or consists of said cavity.

3. The medical implant according to claim 1, wherein said helical thread extends from said outer surface, said helical thread being solid.

4. The medical implant according to claim 1, wherein said conformal lattice structure comprises a plurality of cells, and said helical thread is formed from an extension of struts or walls of some of said plurality of cells extending away from said outer surface of said bone engaging portion.

5. The medical implant according to claim 1, wherein cells of said conformal lattice structure in at least an outer surface of said bone engaging portion are orientated such that insertion of said medical implant by screwing facilitates the ingress of biological matter into said conformal lattice structure.

6. The medical implant according to claim 5, wherein at least some apertures formed by said cells in said outer surface of said bone engaging portion are orientated to face towards a direction of insertion of said implant, such that insertion of said implant drives biological matter into said at least some apertures.

7. The medical implant of claim 1, comprising a plurality of apertures formed by said cells in said outer surface of said bone engaging portion, said plurality of apertures forming an opening into a channel formed by said cells within said lattice structure that is curved from said inner portion to said outer portion in a direction of rotation of insertion of said screw thread.

8. The medical implant according to claim 4, wherein said cells arranged adjacent to and on a side of said helical thread towards an insertion end of said implant, are configured such that apertures formed by said cells face towards a direction of insertion, such that biological matter compressed by said helical thread on insertion of said insert is pushed into said apertures.

9. The medical implant according to claim 5, wherein at least some apertures formed by said cells in said outer surface are configured such that a portion of a perimeter of said at least some apertures are more remote from an insertion end of said medical implant and extend radially outwards further than a portion of said perimeter of said at least one aperture towards said insertion end.

10. The medical implant according to claim 5, wherein at least some apertures formed by said cells in said outer surface are configured such that said at least some apertures are angled with respect to the rotational direction of insertion, such that a portion of the perimeter of the aperture that follows on rotation extends radially beyond a portion that leads allowing the aperture to scoop biological material into the cell on rotation of said implant.

11. The medical implant according to claim 1, wherein said conformal lattice structure is configured such that a size of cell within said conformal lattice structure varies along a length of said bone engaging portion, from a maximum at one end to a minimum at another end.

12. The medical implant according to claim 2, wherein said inner cavity extends to one end of said bone engaging portion, such that one end of said bone engaging portion is open.

13. The medical implant of claim 1, wherein said lattice structure comprises a plurality of interlinked cells forming a mesh structure, said cells comprising a gyroid form.

14. The medical implant of claim 1, wherein at least a portion of said helical thread comprises a plurality of recesses, said recesses being formed at different axial and circumferential positions in said thread.

15. The medical implant of claim 14, wherein said plurality of recesses are arranged to form at least one spiral groove running through said thread.

16. The medical implant according to claim 1, where said medical implant is a dental implant comprising:
said bone engaging portion; and
a transmucosal portion configured to engage marginal soft tissue and an optional abutment portion.

17. A combination of a non-transitory data file and an implant as claimed in claim 1 for replacing a tooth or filling an oral cavity void of a subject, wherein the implant is manufactured by a computer-aided manufacturing process in accordance with information contained in the data file, wherein the data file comprises information relating to physical characteristics of the tooth to be replaced or the void to be filled.

18. The combination according to claim 17, wherein said data file comprises information relating to: (1) a geometrical area and alignment of the tooth to be replaced and neighbouring teeth or geometrical area and alignment of the void; (2) bone mineral density and/or bone quality of a mandibular bone of the subject; and (3) height of a cortical bone of the subject.

19. A method of manufacturing a medical implant according to claim 1, comprising:
determining the bone mineral density and/or bone quality of a subject at a site of insertion of said medical implant;
manufacturing by additive manufacturing a medical implant comprising a bone engaging portion comprising a helical thread extending around an outer surface, said bone engaging portion comprising or consisting of a conformal lattice structure, at least one of a void fraction and a cell size of said conformal lattice structure being selected in dependence upon said determined mineral bone density of said subject.
